SEQUENCE LISTING (4 sequences)

SEQ ID NO: 1 - protein, 258 aa, Homo sapiens
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 2 - protein, 272 aa, Homo Sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 3 - DNA, 1955 bp, Homo Sapiens
gtgccctccgccgctcgcccgcgcgcccgcgctccccgcctgcgcccagcgccccgcgcccgcgccccagtcctcgggcggtcatgctgcccctctgcctcgtggccgccctgctgctggccgccgggcccgggccgagcctgggcgacgaagccatccactgcccgccctgctccgaggagaagctggcgcgctgccgcccccccgtgggctgcgaggagctggtgcgagagccgggctgcggctgttgcgccacttgcgccctgggcttggggatgccctgcggggtgtacaccccccgttgcggctcgggcctgcgctgctacccgccccgaggggtggagaagcccctgcacacactgatgcacgggcaaggcgtgtgcatggagctggcggagatcgaggccatccaggaaagcctgcagccctctgacaaggacgagggtgaccaccccaacaacagcttcagcccctgtagcgcccatgaccgcaggtgcctgcagaagcacttcgccaaaattcgagaccggagcaccagtgggggcaagatgaaggtcaatggggcgccccgggaggatgcccggcctgtgccccagggctcctgccagagcgagctgcaccgggcgctggagcggctggccgcttcacagagccgcacccacgaggacctctacatcatccccatccccaactgcgaccgcaacggcaacttccaccccaagcagtgtcacccagctctggatgggcagcgtggcaagtgctggtgtgtggaccggaagacgggggtgaagcttccggggggcctggagccaaagggggagctggactgccaccagctggctgacagctttcgagagtgaggcctgccagcaggccagggactcagcgtcccctgctactcctgtgctctggaggctgcagagctgacccagagtggagtctgagtctgagtcctgtctctgcctgcggcccagaagtttccctcaaatgcgcgtgtgcacgtgtgcgtgtgcgtgcgtgtgtgtgtgtttgtgagcatgggtgtgcccttggggtaagccagagcctggggtgttctctttggtgttacacagcccaagaggactgagactggcacttagcccaagaggtctgagccctggtgtgtttccagatcgatcctggattcactcactcactcattccttcactcatccagccacctaaaaacatttactgaccatgtactacgtgccagctctagttttcagccttgggaggttttattctgacttcctctgattttggcatgtggagacactcctataaggagagttcaagcctgtgggagtagaaaaatctcattcccagagtcagaggagaagagacatgtaccttgaccatcgtccttcctctcaagctagccagagggtgggagcctaaggaagcgtggggtagcagatggagtaatggtcacgaggtccagacccactcccaaagctcagacttgccaggctccctttctcttcttccccaggtccttcctttaggtctggttgttgcaccatctgcttggttggctggcagctgagagccctgctgtgggagagcgaagggggtcaaaggaagacttgaagcacagagggctagggaggtggggtacatttctctgagcagtcagggtgggaagaaagaatgcaagagtggactgaatgtgcctaatggagaagacccacgtgctaggggatgaggggcttcctgggtcctgttccctaccccatttgtggtcacagccatgaagtcaccgggatgaacctatccttccagtggctcgctccctgtagctctgcctccctctccatatctccttcccctacacctccctccccacacctccctactcccctgggcatcttctggcttgactggatggaaggagacttaggaacctaccagttggccatgatgtcttttct

SEQ ID NO: 4 - DNA, 1722 bp, Homo sapiens
ggggaaaagagctaggaaagagctgcaaagcagtgtgggctttttccctttttttgctccttttcattacccctcctccgttttcacccttctccggacttcgcgtagaacctgcgaatttcgaagaggaggtggcaaagtgggagaaaagaggtgttagggtttggggtttttttgtttttgtttttgttttttaatttcttgatttcaacattttctcccaccctctcggctgcagccaacgcctcttacctgttctgcggcgccgcgcaccgctggcagctgagggttagaaagcggggtgtattttagattttaagcaaaaattttaaagataaatccatttttctctcccacccccaacgccatctccactgcatccgatctcattatttcggtggttgcttgggggtgaacaattttgtggctttttttcccctataattctgacccgctcaggcttgagggtttctccggcctccgctcactgcgtgcacctggcgctgccctgcttcccccaacctgttgcaaggctttaattcttgcaactgggacctgctcgcaggcaccccagccctccacctctctctacatttttgcaagtgtctgggggagggcacctgctctacctgccagaaattttaaaacaaaaacaaaaacaaaaaaatctccgggggccctcttggcccctttatccctgcactctcgctctcctgccccaccccgaggtaaagggggcgactaagagaagatggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaagcgtggcatctgctggtgcgtggacaagtacgggatgaagctgccaggcatggagtacgttgacggggactttcagtgccacaccttcgacagcagcaacgttgagtgatgcgtccccccccaacctttccctcaccccctcccacccccagccccgactccagccagcgcctccctccaccccaggacgccactcatttcatctcatttaagggaaaaatatatatctatctatttgaggaaaaaaaaaaaaaaaaaaaa

(19) United States
(12) Patent Application Publication
(10) Pub. No.: US 2004/0228881 A1
(43) Pub. Date: Nov. 18, 2004

(54) Compositions and methods for modulating neural sprouting

(75) Inventors: Dolly J. Oliver, Cheam (GB); Kei Roger Aoki, Coto De Caza, CA (US); Anton De Paiva, London (GB)

Correspondence Address: Carlos A. Fisher, ALLERGAN, INC., T2-TH, 2525 Dupont Drive, Irvine, CA, 92612, US

(73) Assignees: Allergan, Inc.; Imperial College of Science and Technology

(21) Appl. No.: 10/667,998

(22) Filed: Sep. 18, 2003

Related U.S. Application Data
(63) Continuation of application No. 09/294,980, filed on Apr. 19, 1999, now abandoned.
(60) Provisional application No. 60/083,472, filed on Apr. 29, 1998.

(51) Int. Cl. A61K 39/08; A61K 39/395; A61K 38/18

(52) U.S. Cl. 424/239.1; 514/12; 424/145.1

(57) ABSTRACT

Methods and compositions for modulating neurite outgrowth in damaged neural endplates. Also disclosed are methods for introducing drugs, ribozymes, antisense oligonucleotides and defective receptor genes within neurons.

This application claims priority under 35 U.S.C. §119(e) (1) to provisional application No. 60/083,472, filed Apr. 29, 1998.

### FIELD OF THE INVENTION

The present invention is directed towards methods and compositions for inhibiting neural sprouting in neurons that have been subjected to botulinum toxin. Also disclosed are methods and compositions for extending the period of time during which treatment of nerve cells with botulinum toxin is effective to prevent innervation of a cell or tissue, such as muscle cells or tissue. Such methods and compositions are effective in the treatment of spasms or muscular tetanus. Disclosed as well are methods and compositions for stimulating neural outgrowth.

### BACKGROUND OF THE INVENTION

Neurotoxins, such as those obtained from Clostridium botulinum and Clostridium tetanus, are highly potent and specific poisons of neural cells. These Gram positive bacteria secrete two related but distinct toxins, each comprising two disulfide-linked amino acid chains: a light chain (L) of about 50 KDa and a heavy chain (H) of about 100 KDa, which are wholly responsible for the symptoms of these diseases.

The tetanus and botulinum toxins are among the most lethal substances known to man, having a lethal dose in humans of between 0.1 ng and 1 ng per kilogram of body weight. Tonello et al., Adv. Exp. Med. & Biol. 389:251-260 (1996). Both toxins function by inhibiting neurotransmitter release in affected neurons. The tetanus neurotoxin (TeNT) acts mainly in the central nervous system, while botulinum neurotoxin (BoNT) acts at the neuromuscular junction by inhibiting acetylcholine release from the axon of the affected neuron into the synapse, resulting in a localized flaccid paralysis. The effect of intoxication on the affected neuron is long-lasting and has been thought to be irreversible.

The tetanus neurotoxin (TeNT) is known to exist in one immunologically distinct type; the botulinum neurotoxins (BoNT) are known to occur in seven different immunogenic types, termed BoNT/A through BoNT/G. While all of these types are produced by isolates of C. botulinum, two other species, C. baratii and C. butyricum also produce toxins similar to /F and /E, respectively. See e.g., Coffield et al., The Site and Mechanism of Action of Botulinum Neurotoxin in Therapy with Botulinum Toxin 3-13 (Jankovic J. & Hallett M. eds. 1994), the disclosure of which is incorporated herein by reference.

Regardless of type, the molecular mechanism of intoxication appears to be similar. In the first step of the process, the toxin binds to the presynaptic membrane of the target neuron through a specific interaction between the heavy chain and a cell surface receptor; the receptor is thought to be different for each type of botulinum toxin and for TeNT. The carboxy terminus of the heavy chain appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This last step is thought to be mediated by the amino terminus of the heavy chain, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin then translocates through the endosomal membrane into the cytosol.

The last step of the mechanism of botulinum toxin activity appears to involve reduction of the disulfide bond joining the heavy and light chain. The entire toxic activity of botulinum and tetanus toxins is contained in the light chain of the holotoxin; the light chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. TxNT, BoNT/B BoNT/D, BoNT/F, and BoNT/G cause degradation of synaptobrevin 2 (also called vesicle-associated membrane protein (VAMP)), a synaptosomal membrane protein. Most of the VAMP present at the cytosolic surface of the synaptic vesicle is removed as a result of any one of these cleavage events. Each toxin specifically cleaves a different bond.

BoNT/A and /E selectively cleave the plasma membrane-associated protein SNAP-25; this protein is bound to and present on the cytosolic surface of the plasma membrane. BoNT/C cleaves syntaxin, an integral protein having most of its mass exposed to the cytosol. Syntaxin interacts with the calcium channels at presynaptic terminal active zones. See Tonello et al., Tetanus and Botulism Neurotoxins in Intracellular Protein Catabolism 251-260 (Suzuki K & Bond J. eds. 1996), the disclosure of which is incorporated by reference as part of this specification. BoNT/C₁ also cleaves SNAP-25 at a peptide bond next to that cleaved by BoNT/A.

Both TeNT and BoNT are taken up at the neuromuscular junction. BoNT remains within peripheral neurons, and blocks release of the neurotransmitter acetylcholine from these cells. Through its receptor, TeNT enters vesicles that move in a retrograde manner along the axon to the soma, and is discharged into the intersynaptic space between motor neurons and the inhibitory neurons of the spinal cord. At this point, TeNT binds receptors of the inhibitory neurons, is again internalized, and the light chain enters the cytosol to block the release of the inhibitory neurotransmitters 4-aminobutyric acid (GABA) and glycine from these cells. Id.

Because of its specifically localized effects, dilute preparations of BoNT have been used since 1981 as therapeutic agents in the treatment of patients having various spastic conditions, including strabismus (misalignment of the eye), bephlarospasm (involuntary eyelid closure) and hemifacial spasm. See e.g., Borrodic et al., Pharmacology and Histology Botulinum Toxin in Therapy with Botulinum Toxin 3-13 (Jankovic J. & Hallett M. eds. 1994), incorporated by reference herein. Of the seven toxin types, BoNT/A is the most potent of the BoNTs, and the most well characterized. Intramuscular injection of spastic tissue with dilute preparations of BoNT/A has been also used effectively to treat spasticity due to brain injury, spinal cord injury, stroke, multiple sclerosis and cerebral palsy. The extent of paralysis depends on both the dose and dose volume delivered to the target site. Typically, the neurotoxin is administered in a preparation that also contains several non-toxic proteins as well, including hemagglutins and associated glycoproteins that assist in maximizing its stability and presentation to the target motor neuron.

Typically there is a 24 to 72 hour delay between the administration of the toxin and the onset of the clinical effect. Exposure to the toxin causes denervation atrophy. See e.g., Dutton J., Acute and Chronic Effects of Botulinum Toxin in the Management of Blepharospasm, in Therapy with Botulinum Toxin at 199, incorporated by reference herein. Although the therapeutic application of BoNT has been extraordinarily effective, what side effects have been observed are mainly immediate effects associated with the treatment event itself. Peripheral effects causing weakness in adjacent muscles may sometimes occur; these effects usually do not persist beyond 1 to two weeks. The specific manifestations of these effects upon adjacent muscle groups depend upon the particular indication being treated. For example, patients treated for blepharospasm sometimes experience ptosis, and swallowing problems may occur after injection of neck muscles for torticollis.

Other possible consequences of treatment include the potential for overdosage through miscalculation or differences in activity between different preparations of the toxin, generalized fatigue, and the potential for allergic reactions.

A feature of treatment with BoNT/A, and other clostridial neurotoxin types, is that the paralytic action is temporary with symptoms reappearing in patients within a few months after toxin injection. This characteristic has been thought to be associated with the observed sprouting of nascent, synaptically active processes at the neuromuscular junction (NMJ). The production of such sprouts following BoNT/A therapeutic treatment has appeared to contribute to the reinervation of the treated tissue and therefore the need for repeated serial injections of the toxin.

The duration of paralytic action caused by clostridial neurotoxin and the extent of sprouting, appears to depend on the neurotoxin subtype studied and therefore appears to be related to the specific SNARE target cleaved by each neurotoxin. Thus, BoNT/A and BoNT/C₁, which cleave the t-SNARE protein SNAP-25 within one amino acid of each other, cause a long lasting paralysis with a long average sprout length observed. BoNT/F, which cleaves the v-SNARE protein VAMP, causes a paralysis of shorter duration and sprouts of shorter average length. BoNT/E, which cleaves SNAP-25 at a different position than that of BoNT/A and BoNT/C₁ (thereby liberating a SNAP fragment of different size from the plasma membrane), has a short duration period of about 5 days, and virtually no neural sprouting is observed. Without wishing to be bound by theory, these observations suggest that neural sprouting and duration of paralysis are normally related events, and that the cleavage products of BoNT proteolytic digestion (i.e., the liberated fragment or the membrane-bound fragment) can directly or indirectly regulate, or be coregulated with, neural sprouting.

It would therefore be advantageous to design a method whereby the sprouting phenomenon may be uncoupled from duration of therapeutic effect, and thus delayed, blocked or attenuated so as to prolong the effects of injection of tissue with toxin. Although the experiments described herein utilize a BoNT/A preparation, it will be recognized by those of skill in the art that the methods shown herein will be suitable for employment using other clostridial toxins, such as BoNT/B through /G, and TeNT, in which a sprouting pathway can be observed.

Additionally, it would be advantageous to provide herein compositions effective for the inhibition or prevention of the sprouting phenomenon. Such compositions and methods would lessen the need for patients to undergo repeated neurotoxin treatment.

### SUMMARY OF THE INVENTION

The present invention concerns methods for increasing the period of time between therapeutic treatments of neural tissue with a clostridial neurotoxin; thus the method provides a method of increasing the effectiveness of such treatments. A direct advantage of such methods is an increased therapeutic “life”, and a concomitant lessening in the required frequency of treatment of the patient with neurotoxin. Reducing frequency of treatment would provide less opportunity for a patient to experience the side effects described above that are observed following treatment, but which tend to subside long before the effectiveness of the toxin in the target area has subsided. Additionally, reduced frequency of treatment provides less opportunity for miscalculation of dosage amount and other treatment-specific risks.

Accordingly, an aspect of the invention concerns a composition comprising a first agent comprising a clostridial neurotoxin for use as a therapeutic agent and a second agent able to extend the duration of therapeutic benefit of said first agent, wherein the second agent is effective to attenuate the production of nerve terminal sprouts following treatment of a neuromuscular junction with the clostridial neurotoxin.

In a particular embodiment of this aspect of the invention, the first and second agent may comprise a single entity which is provided the patient in a single treatment session. For example, the entity may comprise a single molecule, or a disulfide-linked multichain polypeptide. Additionally or alternatively, the entity may comprise one or more adsorbed or linked heterogroup, such as a small organic molecule or a nucleic acid linked thereto. The entity preferably comprises both the receptor binding and translocation activities of a clostridial heavy chain and an active portion of a clostridial toxin light chain. The light chain may also comprise an auxiliary enzymatic activity, such as a ribonuclease, which specifically cleaves a nucleic acid encoding an intraneuronal factor which is responsible for the expression, activation and/or secretion of neurotrophic factors or cell adhesion molecules. In a preferred embodiment, such an auxiliary activity is provided by a ribozyme. By ribozyme is meant a nucleic acid or nucleic acid analog having a sequence-specific nuclease activity; the construction and use of ribozymes are well known in the art; see e.g., Cech, T., Science 236:1532-1539(1987); Cech, T. R., Curr. Opin. Struct. Biol. 2:605-609 (1992); and Usman et al., Nucleic Acids & Mol. Biol. 10:243-264 (1996), the disclosures of which are hereby incorporated by reference herein. By nucleic acid analog is meant a polymeric molecule able to form a sequence-specific hybrid with a target single-stranded nucleic acid; such analogs may contain modified nucleotides (or ribonucleotides) such as 3′-O methyl nucleotides, phosphorothioate modified nucleotides, methylphosphonate nucleotides, or nucleotide bases separated by a peptide-like bond.

Alternatively, a nucleic acid or nucleic acid analog comprised in the single entity referred to above may be an antisense agent able to selectively bind to a nucleic acid encoding an intraneuronal factor which is responsible for the expression, activation and/or secretion of neurotrophic factors or cell adhesion molecules. This antisense agent may further provide a double-stranded substrate for the action of an intracellular RNAse H activity. Details concerning certain embodiments of these aspects of the invention are contained in e.g., Dolly et al., International Publication No. WO95/32738, entitled Modification of Clostridial Toxins for Use as Transport Proteins and Uherek et al., J. Biol. Chem. 273:8835-8841 (1998). These two references are incorporated by reference as part of the present application.

A nucleic acid moiety linked polypeptide portion of the entity may encode a protein or polypeptide having the ability to be expressed within a neuron and to directly or indirectly regulate the expression, activation and/or secretion of neurotrophic factors or cell adhesion molecules.

In preferred aspects of the invention, the second agent is selected from the group consisting of agents able to compete with, down-regulate, or neutralize the effects of: IGFI, IGF II, a neurotrophic factor, leukemia inhibitory factor, a nerve cell adhesion molecule and neural agrin. In a more preferred aspect of the invention, the neurotrophic factor is selected from the group consisting of: ciliary neurotrophic factor, NT-3, NT-4, and brain-derived neurotrophic factor and/or the nerve cell adhesion molecule is selected from the group consisting of tenascin-C, ninjurin, neural cell adhesion molecule.

Applicants have surprisingly discovered that recovery of neural function following poisoning of nerve terminals with clostridial neurotoxin involves two distinct and apparently coordinated events. First, the poisoned endplate becomes synaptically inactive. Shortly thereafter the endplate elaborates thin nascent axon neural processes. These processes or “sprouts” are synaptically competent after about 14 days following treatment with clostridial neurotoxin. The sprouts continue growing, reaching a maximal length and level of complexity after about 42 days following treatment with neurotoxin. During this time, the endplate remains synaptically inactive.

Secondly, after about 42 days, the sprouts begin to regress, shortening in length and decreasing in complexity. At the same time, the original endplate begins to become synaptically active, undergoing synaptic vesicle turnover. The increase in such turnover reaches that of the unpoisoned endplate after about 91 days post-treatment, at approximately the same time that the sprouting phenomenon has completely regressed, and no sprouts can be observed. These findings are reported in DePaiva et al., Proc. Nat'l. Acad. Sci. 96:3200-3205 (March 1999), which is hereby incorporated by reference herein.

These observations are diametrically opposed to the prevailing wisdom in the art, in which it has largely been assumed that the original endplate is permanently inactivated upon treatment with clostridial toxin, and that all return of synaptic activity is due to extension of sprouts to compensate for the lack of a neurologically functional endplate. However, as indicated, the old paradigm has been shown by the present Applicants to be in error, in that the poisoned endplate regains neurological function over time, while the axon sprouts regress so that after a given time period the nerve terminal appears essentially as it did prior to treatment. Therefore, Applicants have discovered that, far from being a permanent feature, the axonal sprouts assume a temporary role in the rehabilitation of the poisoned endplate.

Although not wishing to be limited by theory, Applicants believe that these results indicate that the two events outlined above are temporally coordinated, in that a blockage of the neural sprouting phenomenon would delay or block the recovery of the functional endplate. Such temporal coordination could be due to the secretion of one or more factor by the damaged neural endplate (or the inactive muscle fiber) that has neurotrophic effects resulting in the formation of neural sprouts; these sprouts may then elaborate a factor (either the same or different from the first factor) which promotes continued sprouting. This factor may be produced during neurite sprouting in amounts sufficient for the reinervation of the neurotoxin-damaged endplate even after treatment with clostridial toxin. Thus, treatment of cells with an agent able to block neural sprouting would also delay or otherwise attenuate the ability of the treated endplate to experience return of neurological function, and in fact may well block such return altogether.

As indicated, the signaling event indicating the initiation of the neural sprouting phenomenon appears to be mediated by a cytokine or other intercellular messenger. One such agent, agrin, appear to be an important player, if not the key molecule, in the formation of the neuromuscular junction in development, and in neuromuscular regeneration. See Ruegg M. A. and Bixby J. L., Trends in Neurol. Sci. 21:22-27 (1998), the disclosure of which is incorporated herein by reference. Agrin appears to be present in a number of isoforms, which result from alternative mRNA splicing. Soluble agrin isolated from synaptic basal lamina extracts (to which it binds following secretion) is able to induce the aggregation of acetylcholine receptors in the postsynaptic portions of muscle cells. Agrin present in motor neuron terminals (n-agrin) contains an insert, relative to other agrin species, in a region termed the B/z region; this insert is important in conferring the ability on n-agrin to aggregate acetylcholine receptors in postsynaptic tissue. Neural agrin is released by the motor-nerve terminal and is believed by Applicants to induce post-synaptic specialization and up-regulation of other factors, such as muscle-diffusable factors, involved in the neural sprouting response.

It is anticipated that methods which interfere with the sprouting phenomenon (such as by the preventing the action of agrin) would delay the return of innervation to cells which are controlled by neurons which have been therapeutically poisoned (i.e., by BoNT or TeNT) or otherwise damaged. This is because, as indicated above, the rate of return of neural function appears to be partly dependent upon the presence of synaptically active sprouts. Additionally, agents to be used in the inhibition of sprouting may very well also delay or prevent the recovery of neural activity of the endplate following poisoning.

Thus utilization of such methods would therefore be expected to extend the effective period of treatment of tissue with a clostridial toxin, by delaying the regeneration of active neuromuscular synapses, both through inhibition of sprouting and of recovery of the poisoned endplate.

### DETAILED DESCRIPTION OF THE INVENTION

This invention is drawn to methods and compositions for increasing the therapeutic effectiveness of treatment of tissue with clostridial neurotoxin. This increase in effectiveness is made possible by the surprising discovery that regeneration of neural tissue damaged by treatment with clostridial neurotoxin is a complex occurrence in which two coordinated events take place.

In the first of these events, the poisoned neuromuscular endplate becomes synaptically inactive, demonstrating no exocytosis of synaptic vesicles and thus no transport of intracellular acetylcholine. In four days after treatment, the endplate begins to form neural sprouts that are shown to release and regenerate synaptic vesicles. These sprouts grow in length and complexity until approximately 42 days following treatment with the neurotoxin; at this point the neural sprouts begin to regress and shorten. At ninety-one days following treatment, the neural sprouts can no longer be seen.

In the second event, simultaneously with the beginning of regression of the neural sprouts, the synaptically inactive endplate begins to regain the ability to release acetylcholine and begin to recycle synaptic vesicles. This ability, which begins at relatively low levels, increases over the time period indicated above. At approximately 91 days following treatment with clostridial neurotoxin the endplate is histologically and synaptically indistinguishable from the condition of the endplate before treatment with clostridial neurotoxin.

These findings indicate that one may therapeutically intervene at one of the major steps of the sprouting phenomenon to prevent or attenuate the neural sprouting as a method of extending the effective period during which tissue treated with the toxin remains paralyzed. In an initial step, the muscle cells surrounding the neural endplate respond, either sensing the inactive muscle or in response to a signal from the poisoned nerve terminal, by producing muscle-derived diffusable factors. The expression of a number of muscle-derived signaling factors appears to be upregulated by muscle inactivation; such factors include insulin-like growth factors (IGF-1 and IGF-2). A factor such as neural agrin is believed to be the initial signal directing the muscle cell to produce the IGF molecules. Reports have demonstrated that IGF I and IGF II effect neurite outgrowth in cultured BoNT/A treated dorsal root ganglia, and also are able to stimulate the initial sprouting response in paralyzed mouse gluteus muscle. See Caroni, P. and Schneider, C. J. Neurosci. 14:3378-3388 (1994) and Caroni, P., et al. J. Cell Biol. 125:893-902 (1994).

Thus, blocking the effects of such muscle derived diffusable factors that positively affect neurite outgrowth and sprouting may attenuate not only clostridial neurotoxin-induced sprouting, but may also delay the eventual recovery of neurotransmission at the poisoned nerve terminals. Such blocking may occur through the use of antibodies specific for the muscle derived diffusable factor in question, or that are common to such muscle derived diffusable factors. Alternatively, there are naturally occurring binding proteins, such as the IGF binding proteins IGF-BP 4 and IGF-BP 5, which can bind to, and therefore block, the neurotrophic effect of such diffusable factors.

IGF-BP 4 has an amino acid sequence (from the amino terminus) of:

| | |
|---|---|
| MLPLCLVAALLLAAGPGPSLGDEAIHCPPCSEEKLA | (SEQ ID NO: 1) |
| RCRPPVGCEELVREPGCGCCATCALGLGMPCGVYTP | |
| RCGSGLRCYPPRGVEKPLHTLMHGQGVCMELAEIEA | |
| IQESLQPSDKDEGDHPNNSFSPCSAHDRRCLQKHFA | |
| KIRDRSTSGGKMKVNGAPREDARPVPQGSCQSELHR | |
| ALERLAASQSRTHEDLYIIPIPNCDRNGNFHPKQCH | |
| PALDGQRGKCWCVDRKTGVKLPGGLEPKGELDCHQL | |
| ADSFRE | |

This is encoded within the nucleotide base sequence. (from 5′ to 3′) of:

| | |
|---|---|
| GTGCCCTCCG CCGCTCGCCC GCGCGCCCGC GCTCCCCGCC TGCGCCCAGC | (SEQ ID NO: 3) |
| GCCCCGCGCC CGCGCCCCAG TCCTCGGGCG GTCATGCTGC CCCTCTGCCT | |
| CGTGGCCGCC CTGCTGCTGG CCGCCGGGCC CGGGCCGAGC CTGGGCGACG | |
| AAGCCATCCA CTGCCCGCCC TGCTCCGAGG AGAAGCTGGC GCGCTGCCGC | |
| CCCCCCGTGG GCTGCGAGGA GCTGGTGCGA GAGCCGGGCT GCGGCTGTTG | |
| CGCCACTTGC GCCCTGGGCT TGGGGATGCC CTGCGGGGTG TACACCCCCC | |
| GTTGCGGCTC GGGCCTGCGC TGCTACCCGC CCCGAGGGGT GGAGAAGCCC | |
| CTGCACACAC TGATGCACGG GCAAGGCGTG TGCATGGAGC TGGCGGAGAT | |
| CGAGGCCATC CAGGAAAGCC TGCAGCCCTC TGACAAGGAC GAGGGTGACC | |
| ACCCCAACAA CAGCTTCAGC CCCTGTAGCG CCCATGACCG CAGGTGCCTG | |
| CAGAAGCACT TCGCCAAAAT TCGAGACCGG AGCACCAGTG GGGGCAAGAT | |
| GAAGGTCAAT GGGGCGCCCC GGGAGGATGC CCGGCCTGTG CCCCAGGGCT | |
| CCTGCCAGAG CGAGCTGCAC CGGGCGCTGG AGCGGCTGGC CGCTTCACAG | |
| AGCCGCACCC ACGAGGACCT CTACATCATC CCCATCCCCA ACTGCGACCG | |
| CAACGGCAAC TTCCACCCCA AGCAGTGTCA CCCAGCTCTG GATGGGCAGC | |
| GTGGCAAGTG CTGGTGTGTG GACCGGAAGA CGGGGGTGAA GCTTCCGGGG | |
| GGCCTGGAGC CAAAGGGGGA GCTGGACTGC CACCAGCTGG CTGACAGCTT | |
| TCGAGAGTGA GGCCTGCCAG CAGGCCAGGG ACTCAGCGTC CCCTGCTACT | |
| CCTGTGCTCT GGAGGCTGCA GAGCTGACCC AGAGTGGAGT CTGAGTCTGA | |
| GTCCTGTCTC TGCCTGCGGC CCAGAAGTTT CCCTCAAATG CGCGTGTGCA | |
| CGTGTGCGTG TGCGTGCGTG TGTGTGTGTT TGTGAGCATG GGTGTGCCCT | |
| TGGGGTAAGC CAGAGCCTGG GGTGTTCTCT TTGGTGTTAC ACAGCCCAAG | |
| AGGACTGAGA CTGGCACTTA GCCCAAGAGG TCTGAGCCCT GGTGTGTTTC | |
| CAGATCGATC CTGGATTCAC TCACTCACTC ATTCCTTCAC TCATCCAGCC | |
| ACCTAAAAAC ATTTACTGAC CATGTACTAC GTGCCAGCTC TAGTTTTCAG | |
| CCTTGGGAGG TTTTATTCTG ACTTCCTCTG ATTTTGGCAT GTGGAGACAC | |
| TCCTATAAGG AGAGTTCAAG CCTGTGGGAG TAGAAAAATC TCATTCCCAG | |
| AGTCAGAGGA GAAGAGACAT GTACCTTGAC CATCGTCCTT CCTCTCAAGC | |
| TAGCCAGAGG GTGGGAGCCT AAGGAAGCGT GGGGTAGCAG ATGGAGTAAT | |
| GGTCACGAGG TCCAGACCCA CTCCCAAAGC TCAGACTTGC CAGGCTCCCT | |
| TTCTCTTCTT CCCCAGGTCC TTCCTTTAGG TCTGGTTGTT GCACCATCTG | |
| CTTGGTTGGC TGGCAGCTGA GAGCCCTGCT GTGGGAGAGC GAAGGGGGTC | |
| AAAGGAAGAC TTGAAGCACA GAGGGCTAGG GAGGTGGGGT ACATTTCTCT | |
| GAGCAGTCAG GGTGGGAAGA AAGAATGCAA GAGTGGACTG AATGTGCCTA | |
| ATGGAGAAGA CCCACGTGCT AGGGGATGAG GGGCTTCCTG GGTCCTGTTC | |
| CCTACCCCAT TTGTGGTCAC AGCCATGAAG TCACCGGGAT GAACCTATCC | |
| TTCCAGTGGC TCGCTCCCTG TAGCTCTGCC TCCCTCTCCA TATCTCCTTC | |
| CCCTACACCT CCCTCCCCAC ACCTCCCTAC TCCCCTGGGC ATCTTCTGGC | |
| TTGACTGGAT GGAAGGAGAC TTAGGAACCT ACCAGTTGGC CATGATGTCT TTTCT | |

IGFBP 5 has an amino acid sequence (from the amino terminus) of:

| | |
|---|---|
| MVLLTAVLLLLAAYAGPAQSLGSFVHCEPCDEKALS | (SEQ ID No. 2) |
| MCPPSPLGCELVKEPGCGCCMTCALAEGQSCGVYTE | |
| RCAQGLRCLPRQDEEKPLHALLHGRGVCLNEKSYRE | |
| QVKIERDSREHEEPTTSEMAEETYSPKIFRPKHTRI | |
| SELKAEAVKKDRRKKLTQSKFVGGAENTAHPRIISA | |
| PEMRQESEQGPCRRHMEASLQELKASPRMVPRAVYL | |
| PNCDRKGFYKRKQCKPSRGRKRGICWCVDKYGMKLP | |
| GMEYVDGDFQCHTFDSSNVE | |

and is encoded within a nucleotide base sequence (from 5′ to 3′) of:

| | |
|---|---|
| GGGGAAAAGA GCTAGGAAAG AGCTGCAAAG CAGTGTGGGC TTTTTCCCTT | (SEQ ID NO: 4) |
| TTTTTGCTCCT TTTCATTAC CCCTCCTCCG TTTTCACCCT TCTCCGGACT | |
| TCGCGTAGAA CCTGCGAATT TCGAAGAGGA GGTGGCAAAG TGGGAGAAAA | |
| GAGGTGTTAG GGTTTGGGGT TTTTTTGTTT TTGTTTTTGT TTTTTAATTT | |
| CTTGATTTCA ACATTTTCTC CCACCCTCTC GGCTGCAGCC AACGCCTCTT | |
| ACCTGTTCTG CGGCGCCGCG CACCGCTGGC AGCTGAGGGT TAGAAAGCGG | |
| GGTGTATTTT AGATTTTAAG CAAAAATTTT AAAGATAAAT CCATTTTTCT | |
| CTCCCACCCC CAACGCCATC TCCACTGCAT CCGATCTCAT TATTTCGGTG | |
| GTTGCTTGGG GGTGAACAAT TTTGTGGCTT TTTTTCCCCT ATAATTCTGA | |
| CCCGCTCAGG CTTGAGGGTT TCTCCGGCCT CCGCTCACTG CGTGCACCTG | |
| GCGCTGCCCT GCTTCCCCCA ACCTGTTGCA AGGCTTTAAT TCTTGCAACT | |
| GGGACCTGCT CGCAGGCACC CCAGCCCTCC ACCTCTCTCT ACATTTTTGC | |
| AAGTGTCTGG GGGAGGGCAC CTGCTCTACC TGCCAGAAAT TTTAAAACAA | |
| AAACAAAAAC AAAAAAATCT CCGGGGGCCC TCTTGGCCCC TTTATCCCTG | |
| CACTCTCGCT CTCCTGCCCC ACCCCGAGGT AAAGGGGGCG ACTAAGAGAA | |
| GATGGTGTTG CTCACCGCGG TCCTCCTGCT GCTGGCCGCC TATGCGGGGC | |
| CGGCCCAGAG CCTGGGCTCC TTCGTGCACT GCGAGCCCTG CGACGAGAAA | |
| GCCCTCTCCA TGTGCCCCCC CAGCCCCCTG GGCTGCGAGC TGGTCAAGGA | |
| GCCGGGCTGC GGCTGCTGCA TGACCTGCGC CCTGGCCGAG GGGCAGTCGT | |
| GCGGCGTCTA CACCGAGCGC TGCGCCCAGG GGCTGCGCTG CCTCCCCCGG | |
| CAGGACGAGG AGAAGCCGCT GCACGCCCTG CTGCACGGCC GCGGGGTTTG | |
| CCTCAACGAA AAGAGCTACC GCGAGCAAGT CAAGATCGAG AGAGACTCCC | |
| GTGAGCACGA GGAGCCCACC ACCTCTGAGA TGGCCGAGGA GACCTACTCC | |
| CCCAAGATCT TCCGGCCCAA ACACACCCGC ATCTCCGAGC TGAAGGCTGA | |
| AGCAGTGAAG AAGGACCGCA GAAAGAAGCT GACCCAGTCC AAGTTTGTCG | |
| GGGGAGCCGA GAACACTGCC CACCCCCGGA TCATCTCTGC ACCTGAGATG | |
| AGACAGGAGT CTGAGCAGGG CCCCTGCCGC AGACACATGG AGGCTTCCCT | |
| GCAGGAGCTC AAAGCCAGCC CACGCATGGT GCCCCGTGCT GTGTACCTGC | |
| CCAATTGTGA CCGCAAAGGA TTCTACAAGA GAAAGCAGTG CAAACCTTCC | |
| CGTGGCCGCA AGCGTGGCAT CTGCTGGTGC GTGGACAAGT ACGGGATGAA | |
| GCTGCCAGGC ATGGAGTACG TTGACGGGGA CTTTCAGTGC CACACCTTCG | |
| ACAGCAGCAA CGTTGAGTGA TGCGTCCCCC CCCAACCTTT CCCTCACCCC | |
| CTCCCACCCC CAGCCCCGAC TCCAGCCAGC GCCTCCCTCC ACCCCAGGAC | |
| GCCACTCATT TCATCTCATT TAAGGGAAAA ATATATATCT ATCTATTTGA | |
| GGAAAAAAAA AAAAAAAAAA AA | |

These binding proteins can be made synthetically or cloned and produced for therapeutic purposes, while a cell line producing a desired monoclonal antibody can be maintained for relatively large-scale antibody production. Cloning and general antibody methodologies are commonplace in the art; such methodologies are disclosed within Sambrook et al., Molecular Cloning: A Laboratory Manual (2nd ed. Cold Spring Harbor Laboratory Press 1989), the disclosure of which is hereby incorporated by reference as part of this disclosure.

Rather than using competitive methods, another aspect of the invention involves the use of a cholinergic special transporter to insert a gene which produces inactive receptors for one or more factor involved in promotion of neural sprouting. Such receptors would maintain high specific binding constants to their ligands, but the biological activity of the receptors would be abrogated; such receptors could be easily be generated and screened through the introduction of mutations in the nucleotide sequence encoding the protein, and assaying the mutants for binding strength and biological activity. Alternatively, the neurotrophic activities of factors produced by the neural endplate or nascent sprouts may be inhibited through intracellular targeting and delivery of a competitive inhibitor, ribozyme, transcriptional suppresser or other agent specifically able to block or attenuate such activities, as described above. Such intracellular targeting is disclosed in references such as, e.g., Dolly et al., International Patent Publication No. WO95/32738, previously incorporated by reference herein.

The second point at which intervention in the sprouting phenomenon may be made is during the stage of axonal outgrowth and arbor development. At this stage such outgrowth has already been initiated, but auxiliary factors appear to be necessary in order to maintain axonal growth. A number of factors are known to affect the rates of outgrowth, and may also effect the survivability of many types of neurons. Such factors include ciliary neurotrophic factor (CNTF); neurotrophins, including NT-3, NT-4, and brain-derived neurotrophic factor (BDNF); and leukemia inhibitory factor (LIF). Not only are these factors important in establishing an initial rate of axonal outgrowth, but they appear to either directly or indirectly stimulate their own production—therefore blocking the initial outgrowth of sprouts may be essential in preventing further propagation through the continued expression of such factors.

The same methods as disclosed above can be used to prevent one or more of the agents listed above to manifest its activity. As would be expected by those of skill in the art in light of the present disclosure, such agents, destroy or bind to, and therefore attenuate the activity of these factors or their block their ability to bind to their receptors, or inactive forms of their receptors, would be useful when used in conjunction with a clostridial neurotoxin to prevent or reduce the rate of sprouting of neurites from the poisoned or damaged endplate.

The third stage at which the sprouting phenomenon may be attenuated or inhibited concerns the binding of axons to the extracellular matrix. Axons are guided to cellular processes containing the appropriate neurotransmitter receptors by binding to components of the extracellular matrix. Such binding involves a variety of cell-borne or matrix associated adhesion molecules. Tenascin-C is an extracellular matrix component derived from Schwann cells that appears to bind neural processes. Ninjurin is a cell surface adhesion molecule that is up-regulated following peripheral nerve injury and thought to be involved in exonal guidance. See Araki, T., et al., J. Biol. Chem. 272:21373-21380 (1997), incorporated by reference herein. Similarly, neural-cell adhesion molecule (N-CAM), is an adhesion molecule which is thought to be involved in binding of neural sprouts to the extracellular matrix. The same techniques indicated above may be employed to prevent the expression or inhibit the activity of these molecules when used as part of a clostridial neurotoxin therapy.

Thus, in one aspect the present invention is drawn to a method for extending the effective period during which tissue treated with clostridial toxin is paralyzed, comprising: Contacting said tissue with a composition comprising an agent able to prevent the neuroregenitive activity of a polypeptide selected from the group consisting of IGF-1, IGF-2, cilary neurotrophic factor, NT-3, NT-4, brain-derived neurotrophic factor, leukemia inhibitory factor, tenascin-C, ninjurin, neural cell adhesion molecule, and neural agrin.

In one preferred embodiment, the agent comprises a polypeptide able to bind to IGF-1 and/or IGF-2 in a manner that prevents an IGF molecule from binding to or activating a cell surface receptor involved in the initiation of neural sprouting. In a most preferred embodiment the polypeptide comprises at least a portion of a amino acid sequence selected from the group consisting of: IGFBP-4 (SEQ ID NO: 1) or IGFBP5 (SEQ ID NO: 2). Preferably said portion comprises at least 10 contiguous amino acids of said sequence; more preferably said portion comprises at least 20 contiguous nucleotides of said sequence. Most preferably, the portion comprises an amino acid sequence selected from the group consisting of the entire amino acid sequence of IGFBP-4 or IGFBP-5.

Treatment of cells with such a composition may be accomplished either before or simultaneously with treatment with clostridial toxin. Preferably, the clostridial toxin is a botulinum toxin. Even more preferably, the botulinum toxin comprises BoNT/A. In other embodiments, the clostridial toxin is TeNT. Agent which are able to bind to any of these factors in a manner that inhibits their neurotrophic activity, or which bind to the receptors for such factors, would, in light of the present application, be expected to function as agents for extending the effective period between treatments of tissue with a neurotoxin.

Another embodiment comprises a cholinergic specific transporter joined to a gene encoding a gene which produces an inactive receptor for one or more factor involved in promotion of neural sprouting when delivered to a neural cell in vivo. Preferably the receptors maintain high specific binding constants to said factor(s) and the biological activity of the receptor is reduced or absent. In a particularly preferred embodiment the transporter comprises some or all of a clostridial neurotoxin heavy chain, although other transporters such as the diphtheria toxin transporter may be effective in this regard as well.

In another embodiment the invention comprises a cholonergic specific transporter that is covalently or non-covalently joined to a nucleic acid which comprises a ribozyme or antisense nucleic acid able to specifically destroy the nucleic acids encoding neurotrophic agents or their receptors. Said joining may be made through methods including, but not limited to, covalent bonding or electrostatic forces.

In another embodiment, the present invention is drawn to the methods for stimulating the outgrowth of neural sprouts from damaged neural tissue. Such methods could be effective ways of increasing the rate at which reinnervation occurs after a neural injury. These methods comprise: Contacting said tissue with a composition comprising a polypeptide which comprises a neurotrophically active domain derived from an agent selected from a group consisting of IGF-1, IGF-2, cilary neurotrophic factor, NT-3, NT-4, brain derived neurotrophic factor, leukemia inhibitory factor, tenascin-C, ninjurin, neural cell adhesion molecule, and neural agrin. Such damage may be a result of neurotoxin poisoning or due to a traumatic event, including but not limited to nerve or spinal cord crush injuries, traumatic brain injuries, glaucoma-induced damage to the retina and/or optic nerve, or surgical trauma or injury.

The following examples illustrate various embodiments of the present invention, and are not intended to limit the scope of the invention, which is solely defined by the claims which conclude this specification.

### EXAMPLE 1

Blepharospasm is a medical condition characterized by uncontrolled eyelid movement. In its early stages, the condition is characterized by excessive blinking or fluttering of the eyelids. The condition is generally a progressive one, in which excessive blinking is replaced in the later stages with spasms of eye closure that interfere with visual function. The spasms become more frequent and severe, and involve the preseptal, pretarsal, and orbicularis oculi muscle. The condition often results in functional blindness relatively quickly (in a matter of two to three years) after the symptoms are first encountered.

A patient suffering from moderate idiopathic blepharospasm is treated with injections of BoNT/A toxin preparation containing non-toxic proteins and hemagglutins in sterile saline. Alternatively, the same toxin preparation without hemagglutins may be used. The injections are generally in the volume of 100 μl; and each injection contains 1.25 to 2.5 units of the toxin preparation. The injections are made into the pretarsal orbicularis oculi of the upper lid laterally and medially and in the lower lid laterally and medially. Additionally, 2.5 unit injections (100 μl each) are made lateral to the lateral canthus and into the brow medially. Total amount of BoNT/A toxin injected is roughly 6.25 to 12.5 units per eye. The BO/A toxin is provided in a sterile, preservative-free saline, and the same solution is used to dilute the BoNT/A toxin if the master preparation of it is too concentrated.

Following injection the treated muscles are sufficiently paralyzed due to this treatment to alleviate the major symptoms of blepharospasm. Some mild concomitant weakness in the surrounding muscle tissue is observed; these side effects are mild and tolerated well by the patient. The effect of this treatment lasts approximately 8 weeks, and must be repeated at the end of this time to maintain the beneficial effects.

### EXAMPLE 2

A patient with blepharospasm is pre-treated with BoNT/A toxin as indicated in Example 1 with the following difference. Prior to injection with BoNT/A toxin preparation, the patient is given a ten-fold excess of IGFBP-4, having an amino acid sequence of SEQ ID NO: 1. The binding protein preparation is dissolved in sterile, preservative-free saline. Each injection is in the same area as the toxin injections that follow the pre-treatment; the volume of each injection is 100 μl. The BoNT/A toxin preparation is injected ten minutes after the injection of the IGFBT 4 injection.

The patient's therapeutic response to the BoNT/A toxin is similar to that seen in Example 1. The duration of the benefit provided by the BoNT/A toxin treatment is extended to 12 weeks or more, during which time no further injection need be made.

### EXAMPLE 3

A patient with blepharospasm is pre-treated with BoNT/A toxin as indicated in Example 1 with the following difference. The BoNT/A toxin has been modified to have joined thereto a nucleic acid comprising a ribozyme specifically targeted to enzymatically destroy neural agrin mRNA. No supplemental injections are made.

The patient's therapeutic response to the BoNT/A toxin is similar to that seen in Example 1. The duration of the benefit provided by the BoNT/A toxin treatment is extended to 12 weeks or more, during which time no further injection need be made.

### EXAMPLE 4

A patient with blepharospasm is pre-treated with BoNT/A toxin as indicated in Example 1 with the following difference. The BoNT/A toxin has been modified to have joined thereto a nucleic acid encoding an inactive neurotrophin receptor which retains the ability to bind its target neurotrophin. No supplemental injections are made.

The patient's therapeutic response to the BoNT/A toxin is similar to that seen in Example 1. The duration of the benefit provided by the BoNT/A toxin treatment is extended beyond that seen with BoNT/A alone, during which time no further injection need be made.

It will be understood that, while reference is made to BoNT/A in the Examples above, any other of the species of botulinum toxins (e.g., BoNT/B through G) could be substituted therefor, with appropriate adjustments possibly necessary due to differences in specific activity of the toxin. Additionally, the light chain segment could be derived from any clostridial neurotoxin (or other neurotoxin), with the heavy chain retaining the motor neuron receptor binding and exo-vescular transport activities retained from the BoNT heavy chain.

These Examples are not intended to exhaust the embodiments of the present invention, and the invention is not to be seen as limited thereby. Further embodiments will be disclosed within the claims that conclude this specification.

What is claimed is:

1) A method for extending the effective period during which tissue treated with a clostridial toxin is paralyzed comprising: contacting said tissue with a composition comprising an agent able to prevent the neuroregenerative activity of a polypeptide selected from the group consisting of: IGF I, IGF II, cilary neurotrophic factor, NT-3, NT-4, brain-derived neurotrophic factor, leukemia inhibitory factor, tenascin-C, ninjurin, neural cell adhesion molecule, and neural agrin.

2) The method of claim 1 wherein said contacting step occurs at the same time as said tissue is treated with said clostridial toxin.

3) The method of claim 1 wherein said contacting step occurs prior to treatment of said tissue with said clostridial toxin.

4) The method of claim 1 wherein said clostridial toxin comprises BoNT.

5) The method of claim 1 wherein said clostridial comprises BoNT/A.

6) The method of claim 1 wherein said agent is selected from the group consisting of:
a) an antibody able to selectively bind said polypeptide,
b) a competitive inhibitor of said polypeptide,
c) a compound able to selectively prevent the expression of a gene encoding said polypeptide,
d) a binding protein other than an antibody, and
e) a ribozyme,
f) a nucleic acid encoding an inactive growth factor receptor able to bind said growth factor.

7) The method of claim 6 wherein said agent is an antibody able to selectively bind said polypeptide.

8) The method of claim 6 wherein said agent is a competitive inhibitor of said polypeptide.

9) The method of claim 6 wherein said agent is a compound able to prevent the expression of a gene encoding said polypeptide.

10) The method of claim 6 wherein said agent is a binding protein other than an antibody.

11) The method of claim 9 wherein said polypeptide is selected from the group consisting of IGF I and IGF II, and said binding protein is selected from the group consisting of IGF-BP4 and IGF-BP5.

12) A method for stimulating the outgrowth of neural sprouts from damaged neural tissue comprising: contacting said tissue with a composition comprising a polypeptide which comprises a neurotropically active domain derived from an agent selected from the group consisting of IGF I, IGF II, cilary neurotrophic factor, NT-3, NT-4, brain-derived neurotrophic factor, leukemia inhibitory factor, tenascin-C, ninjurin, neural cell adhesion molecule, and neural agrin.

13) The method of claim 11 wherein said agent comprises IGF I.

14) The method of claim 11 wherein said agent comprises IGF II.

15) The method of claim 11 wherein said agent comprises NT-3.

16) The method of claim 11 wherein said agent comprises ciliary neurotrophic factor.

17) The method of claim 11 wherein said agent comprises NT-3.

18) The method of claim 11 wherein said agent comprises NT-4.

19) The method of claim 11 wherein said agent comprises brain-derived neurotrophic factor.

20) The method of claim 11 wherein said agent comprises leukemia inhibitory factor.

21) The method of claim 11 wherein said agent comprises tenascin-C.

22) The method of claim 11 wherein said agent comprises ninjurin.

23) The method of claim 11 wherein said agent comprises neural-cell adhesion molecule.

24) The method of claim 11 wherein said agent comprises neural agrin.
